(19) United States
(12) Patent Application Publication
(10) Pub. No.: US 2022/0175349 A1
(43) Pub. Date: Jun. 9, 2022

(54) System and Methods for Ultrasound Imaging with Modularized Frontend and Personal Computer System

(71) Applicant: Yanwei Wang, Bellevue, WA (US)

(72) Inventor: Yanwei Wang, Bellevue, WA (US)

(21) Appl. No.: 17/112,553

(22) Filed: Dec. 4, 2020

(51) Int. Cl. A61B 8/00; A61B 8/08; A61N 7/00

(52) U.S. Cl. CPC A61B 8/565; A61B 8/5207; A61N 2007/0052; A61B 8/54; A61N 7/00; A61B 8/42

(57) ABSTRACT

A modularized ultrasound apparatus utilizes a PC system such as PC case, thermal management subsystem, power supply unit, motherboard, CPU, memory, hard drive, GPU, to build an ultrasound system by inserting frontend modules integrated on PCIe expansion cards as modularized components into the PC system's PCIe expansion subsystem.

### FIELD OF THE INVENTION

The present invention relates to the field of ultrasound imaging apparatus, systems, and methods for imaging and/or treatment. Specifically, the present invention relates to reducing cost, improving scalability, improving flexibility, improving computational power, of ultrasonic diagnostic or treatment apparatus.

### BACKGROUND OF THE INVENTION

From development cost point-of-view, to develop an ultrasound imaging system with premium capability from scratch, a company/person faces the challenges of high development cost associated with building teams of 1) electronic hardware engineers to design and produce analog/digital circuits, 2) firmware engineers to drive these circuits and chips, 3) software engineers to work on various part of the system such as implementation of image reconstruction and processing algorithms, program control path, design UI, etc, 4) mechanical engineers to develop various modules of the system, such as system chassis/box and assemble the parts together.

From functional point-of-view, an ultrasound apparatus can be divided into two separate systems. 1) Frontend subsystem responsible for: transmit signal generation, receive signal conditioning and sampling, coordinating transmit and receive, send ADC sampled data back to imaging processor. 2) Imaging processor subsystem is responsible for beamforming, mid-end signal conditioning and processing, back-end image processing to generate the image ready for display. Diagrams of such architectures are available online. See for example, https://www.ti.com/solution/ultrasound-scanner, for various designs.

Existing ultrasound imaging devices developed by current manufacturers are closed systems with proprietary hardware components, proprietary firmware interface, complex software system which is not an option for small company or researcher to develop their own ultrasound system upon. Such existing system has high hardware/manufacture cost, high development cost due to the teams of engineers worked together to develop such complex and customized system. Such existing system also has limited computation power due to the limited size of the system, or the choice of GPU model or number of GPUs, and not upgradable by the customer to the latest technology on the market.

Another option is research ultrasound. Research ultrasound is an ultrasound box sitting outside of a computer and connected to the computer via high-speed links such as PCIe or USB3.0. The box either includes ultrasound front-end only which sends pre-beamformed data back to the computer, or together with beamforming module which sends beamfored data back to the computer, or with post-processing module as well which sends post-processed display ready data back to the computer. Research ultrasound provides the opportunity for developer to design and implement their own specialized ultrasound system. Due to the lack of using off-the-shelf components and not unified system architecture, such platform usually have high hardware/manufacturing cost, bulky appearance, separated parts of data acquisition unit and data processing unit loosely connected to make the whole system.

The third option is OEM ultrasound which is an ultrasound box integrated with frontend as well as beamforming and post processing. The ultrasound box will need to connect to a PC. This approach presented a few problems too: 1. as with the case of research ultrasound, the two-box design make the system less compact and special hardware may be needed to secure and combine them together in case one combined system is desired. (The PC box can take the form factor of either a regular PC box, a laptop, or a small form factor PC). 2. not scalable to configure an arbitrary high number of channels for the system: When required channel counts need multiple boxes, it's not possible to combine multiple box together to produce the final product. 3. When other customized hardware needed, it can be plugged into the PC's extension slot. This leads to a large size PC box needed which will be paired with the ultrasound box to build the complete system. For this case, the PC can be configured with high computational power such as GPUs, since the beamforming and post processing has already done in the OEM box, there is not much can be done to develop your own ultrasound imaging system if novel beamforming or post processing are need to be implemented.

All the existing approaches above does not provide some or all of the following aspects for an ultrasound imaging system: 1) low cost, 2) enough flexibilities of system size and shape, 3) enough scalability to build high channel count systems, 4) high (or almost unlimited) computational power.

Therefore, what is needed are systems and methods that overcome the above mentioned disadvantages and provide an apparatus which provides the best of all the aspects mentioned above.

### SUMMARY OF THE DESCRIPTION

Recent introduction of GPU into the ultrasound industry has the opportunity to significantly reduce the hardware and development cost to develop an ultrasound system. As the main processing unit, compared to the alternative technologies (previous approaches) using ASIC, FPGA, specialized DSP, and CPU, GPU can reduced the cost by magnitudes due to its extremely low hardware cost as an off-the-shelve consumer product and low development cost as only software needs to be implemented. For example, the ultrasound beamformer can be implemented by using off-the-shelve consumer grade GPU with magnitude of cost reduction compare with industry grade ASIC, FPGA, DSP, not to mention that the reduction of development cost is even more. It opens up extremely fast approaches to develop new ways to implement beamforming, image reconstruction, new imaging mode, other innovations, etc. It also provides a convenient low cost way to extend system's computational power by adding additional GPU cards, or simply upgrade to the latest GPU model.

Recent advances of the ultrasound frontend integrated circuits (IC) with highly integrated off-the-shelf frontend chips for transmit, receive, and analog to digital converter (ADC) make the development of compact ultrasound frontend module within a small form factor such as a PCIe card become feasible. The prevalence of PCIe hard IP on low cost FPGA makes the low cost frontend card possible. Now it is practical to produce low cost, highly integrated frontend module in a PCIe card/module form factor, and assemble an ultrasound system by plugging PCIe frontend cards/modules into the motherboard PCIe extension slots and using the existing PC chassis/case to hold all the components needed without design and manufacture proprietary system chassis.

The present inventor has 1) observed above evolutions; 2) realized that to build a premium capable ultrasound system, the only parts needs to be built is the Frontend: Image Processor and other peripheral subsystems can be assembled via off-the-shelve components; and 3) invented a modularized way to develop ultrasound system with extremely low hardware and development cost, as well as extremely high computational power. With modularly designed hardware and software, current invention provides a fast and convenient way to develop ultrasound imaging systems with: best flexibility of choosing system form factor, size, and shape; best scalability to extend to higher channel counts; lowest cost by maximum using off-the-shelve consumer components; best computational power by free choice of the latest GPU model and number of GPUs.

To achieve this claim, an ultrasound frontend module is developed as a PCIe extension card which can be inserted into a motherboard PCIe slots and sit inside the PC case without requiring an extra box to hold it outside of PC. One or multiple frontend module can work together as a frontend subsystem to achieve various physical channel configurations. An ultrasound probe can be connected directly to the connector sitting on the frontend card's PCI bracket, or through a probe connector adaptor/converter, especially when multiple frontend cards are involved. Closed shielding enclosure is used for the frontend card to avoid environmental EM interferences inside or outside of PC case. The cooling fans of the PC chassis/case's thermal system and the cooling fans/heat sink on the Frontend module work as the thermal management system to keep the Frontend and the whole system from overheat. The ultrasound pre-beamformed data collected by the frontend will be DMA transferred to CPU or GPU memory through PCIe bus. Power regulators are used to convert the 12V or 5V power output from the power supply unit (PSU) into various analog and digital voltage rails used by the Frontend module. These power regulators can be located either on each Frontend module or on a separate Frontend Power Supply Module (FPSM).

A few choices are available to configure the implementation of the beamforming and backend processing according to the configuration of the system. For system without GPU, both beamforming and backend can be implemented on CPU. For system with GPU, beamforming as well as other backend processing can be implemented on GPU. Another option with GPU is to partition the processing algorithms between GPU and CPU.

The invented platform also enables a new market: individual who is not an expert in developing ultrasound machines can develop their own state-of-the-art ultrasound system by plugging in frontend cards and connect an ultrasound probe. The standard accessories and modularized hardware and software enables developers to develop or customize their own systems by off-the-shelve components. There are significant flexibility of the system developed by using the disclosed platform. For example, when multiple frontend cards are used, the clock and trigger signals from the master needs to be connected to other slaves, which can be solved by using off-the-shelve Y cables.

With PCIe card based frontend module as off-the-shelf component, developer of ultrasound system can simply choose a few frontend cards, two for example, plug them into the existing PC case, also plug-in a GPU card for high computation power and good system performance, to finish up the hardware development of the system.

One easy way to extend the system to high channel count system by using off-the-shelve PCIe enclosures. One PCIe enclosure can be used to hold multiple Frontend modules, hence is called a high channel count module (HCCM). One PCIe enclosure can be used to hold multiple GPUs. To scale the system in both physical channels and computational power, we just need to increase the number of HCCM and GPU enclosures.

Pre-beamformed data of all the physical channels are sent to host in real time, a large amount of DMA buffers needed inside host memory. These DMA buffers can be Pre-Allocated during the boot-up time of the system.

Large amount pre-beamformed data of all the physical channels are sent to host in high bandwidth, a significant amount of time in GPU is receiving the data through copy from host. To better utilize GPU, asynchronies mode are used to overlap the data copy time and the execution of imaging processing algorithms to hide this data copy time.

To meet the requirements of medical device regulation, medical degree off-the-shelve power supply unit can be used for this purpose and avoid delay to the market.

With low cost frontend module and usage of mass produced off-the-shelf consumer electronic components such as GPU, PC chassis/case, motherboard, memory and hard drive, power supply, PCIe enclosure, etc, the disclosed ultrasound system can simultaneously achieve previously conflicting advantages: 1 low hardware cost, 2 low development cost, 3 great flexibility in choosing system form factor with existing off-the-shelf cases and PCIe enclosures, 4 easy to extend to high channel count systems (higher than conventional 128/256 channel) by using existing PCIe enclosures, 5 high computational power and easily upgradable to latest technology.

### BRIEF DESCRIPTION OF THE DRAWINGS

The present invention is illustrated by way of example and not limitation in the figures of the accompanying drawings in which like references indicate similar elements.

FIG. 1 illustrates a Frontend PCIe module, according to one embodiment of the present invention.

FIG. 2 illustrates a completely assembled system, according to one embodiment of the present invention.

FIG. 3 illustrates a top view diagram of an embodiment of a high channel count module (HCCM).

FIG. 4 illustrates another completed system with high physical channel count and high computation power, according to one embodiment of the present invention.

FIG. 5 illustrates PCB design of the frontend PCIe card, according to one embodiment of the present invention.

FIG. 6 illustrates FPGA frontend control system design block diagram, according to one embodiment of the present invention.

FIG. 7 illustrates a diagram of the master high channel count module (HCCM)'s clock and synchronization system. A HCCM subsystem configured with multiple frontend PCIe modules interconnected, according to one embodiment of the present invention.

FIG. 8 illustrates a diagram of the slave high channel count module (HCCM)'s clock and synchronization system.

FIG. 9 illustrates an embodiment of the probe connector convertor, which connects two Frontend Modules and one ultrasound probe, according to one embodiment of the present invention.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described in order to provide a concise discussion of embodiments of the present inventions.

Reference in the specification to “one embodiment” or “an embodiment” or “another embodiment” means that a particular feature, structure, or characteristic described in conjunction with the embodiment can be included in at least one embodiment of the invention. The appearances of the phrase “in one embodiment” in various places in the specification do not necessarily all refer to the same embodiment.

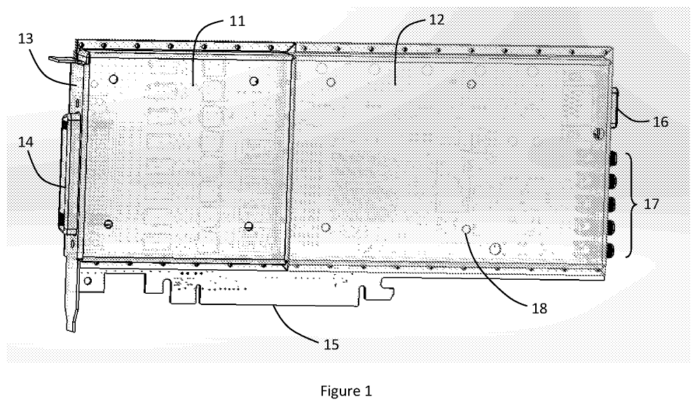

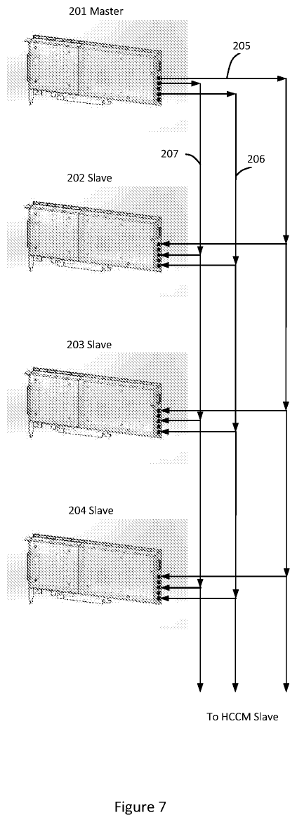

FIG. 1 illustrates diagram of an embodiment of a Frontend Card (Module): transmitter, receiver amplifier, A/D converter, FPGA controller, memory block, Clock circuitry, power regulator, all located on a PCIe card form factor, with a high element count connector 14 exposed outside of the PCIe bracket 13. The PCIe gold finger is shown as 15, Shield boxes for analog and digital circuits are shown as 11 and 12, respectively. The power connector 16 is used to connect to an outside power supply. A group of 5 coax connectors 17 are used for clock signal input/output, synchronous trigger signals input/output. Note that as shown in the figure, the PCI bracket and the shielding box are generated from the same piece of sheet of shielding material. Note that in FIG. 7 below, a Frontend subsystem with multiple Frontend Cards (Modules) are illustrated by inter-connecting these Frontend Cards through these coax connectors.

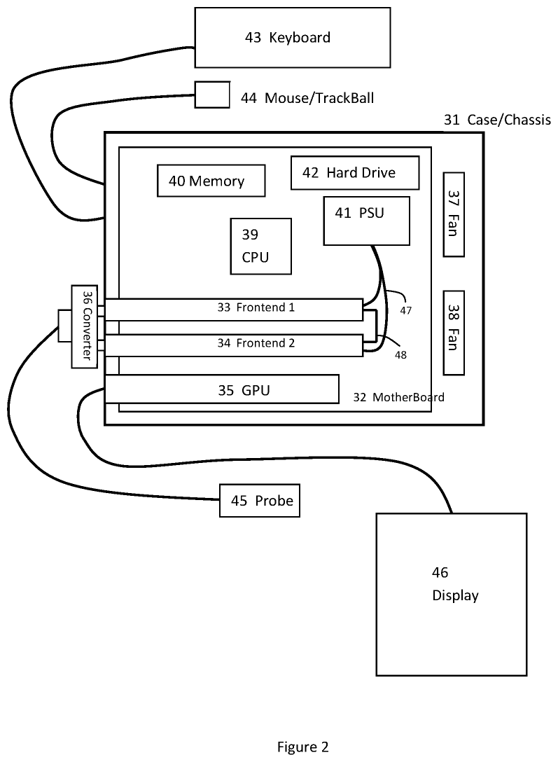

FIG. 2 illustrates top view diagram of an embodiment of a completely assembled system for implementing an apparatus of the present disclosure: a complete PC system with two Frontend modules plugged into its motherboard high-speed extension slots. All the standard parts for a PC system are included, such as a case 31, power supply unit (PSU) 41, cooling fans 37 and 38, hard drive 42, motherboard 32, GPU 35, CPU 39, Memory 40, display 46, keyboard 43, trackball 44. Only non existing off-the-shelve components are the two Frontend Modules 33, 34 and the probe connector 36.

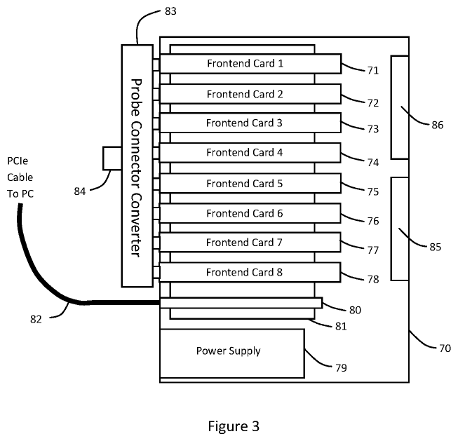

FIG. 3 illustrates a top view diagram of an embodiment of a high channel count module (HCCM) which is implemented by a PCIe extension enclosure 70. In the diagram, there are 8 PCIe Frontend modules are used, 71 to 78. All these Frontend modules are plugged in the PCIe slots on the extension board 81 inside the enclosure 70. An upstream adapter 80 is commonly used to connect the PCIe enclosure system to a Host via PCIe extension cable 82. Such enclosures has a power supply unit 79. A probe connect adaptor/converter 84 is configured to connect to all the Frontend modules at one side, and at the same side, connect to one or multiple probes.

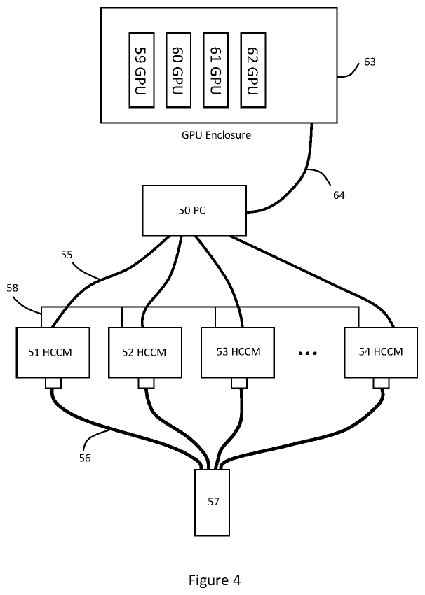

FIG. 4 illustrates a diagram of an embodiment of a complete high channel count system with one or multiple high channel count modules (HCCM) 51˜54: all the extension enclosures are connected to the PC with high-speed bus 55 such as PCIe or Thunderbolt. Probe connection cables 56 are used to connect probe 57 to each HCCM. A clock and sync signal coax network 58 is used to interconnect all the HCCMs. For example, if each Frontend module has 64 physical channels, each HCCMs will have 256 physical channels, and the 4 HCCMs system in FIG. 4 will have 1024 physical channels. Clearly, a 2048 physical channel system can be build by using 8 HCCMs, etc. A high process power module (HPPM) 63 is illustrated in the system: a PCIe enclosure 63 is used to host 4 GPUs 59˜62. HPPM is connected to the PC with PCIe or thunderbolt cable 64.

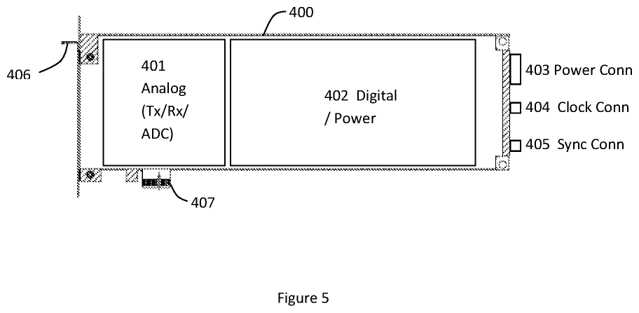

FIG. 5 illustrates the PCB layout of an embodiment of the Frontend card. The PCB 400 is in a PCIe form factor, where the analog circuit 401 such as transmit, receive, high voltage switch, and ADCs are located close to the PCIe bracket 406, which is shown in FIG. 1 as item 13. The digital circuit 402 are located at the opposite side of the PCIe bracket 406. Power connector 403, clock input/output connector 404, and synchronization signal input/output connector 405 are shown. PCIe gold finger 407.

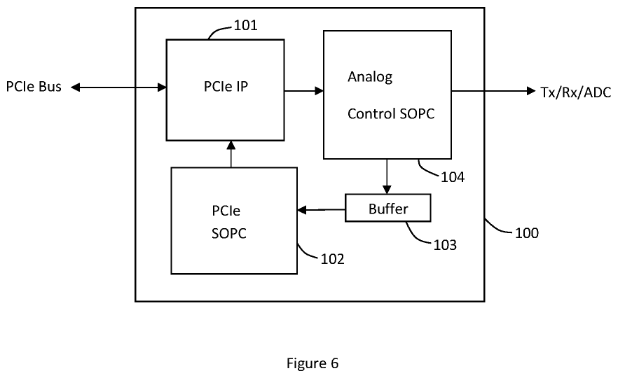

FIG. 6 illustrates a diagram of an embodiment of the frontend FPGA control system 100 in the Frontend module. A PCIe hardiP 101 is used to connect to the system PCIe bus to receive and send data between Frontend module and CPU or GPU. An analog control system-on-a-programmable-chip (SOPC) 104, also known as soft CPU, can receive configurations of an imaging mode from CPU through PCIe hardIP, and generate each transmit, receive, ADC, and save the data into a buffer 103. An PCIe SOPC 102 is configured to access the common buffer 103, generate DMA sequence and drive the PCIe hardIP 101 in the FPGA to send the DMA data back to CPU/GPU memory through system PCIe bus. The analogy control 104 and the PCIe control 102 are designed to use separate clock domains. As a bridge, the common buffer 103 can be accessed by both Analog control SOPC 104 and PCIe control 102, in a form of dual port memory.

FIG. 7 illustrates a diagram of an embodiment of the Master HCCM's synchronization system. One Frontend Module 201 is either hardware or software configured as Master and the other three Frontend Model 202˜204 are configured as slaves. The master card will output clock signal 205 which is sent to other slave modules to use. Master card will also generate frame trigger 206 signals which trigger slave cards to start a new frame. Master card will also generate a line trigger 207 which trigger slave cards to start a new transmit and receive event. All the Frontend cards will send their received data individually to the CPU/GPU memory. The off-the-shelve coax cable or Y cable could be used for these interconnection between Master and Slaves.

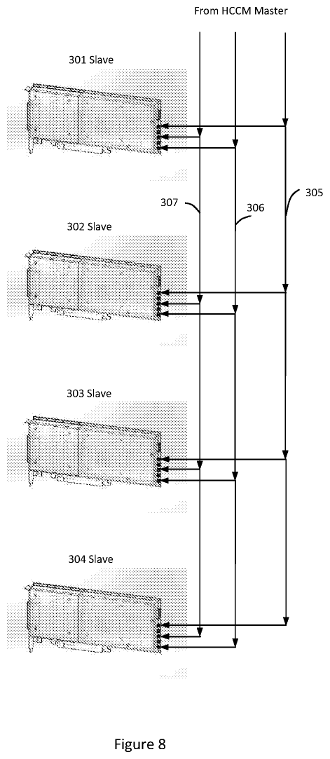

FIG. 8 illustrates a diagram of an embodiment of the Slave HCCM's synchronization system. All the Frontend Modules 301˜304 are slave. The clock 305 and trigger signals 306,307 are all from a Master HCCM.

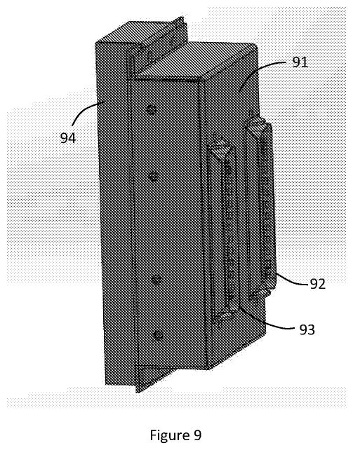

FIG. 9 illustrates a diagram of an embodiment of the probe adapter (probe connector convertor) 91. two high element count connectors 92 and 93 are used to connect to two Frontend modules. A ultrasound probe connector 94 are located on the other side of the probe connector converter. Proper shielding materials are used to enclose these connectors.

1. An ultrasound imaging apparatus comprising:
a frontend subsystem configured to control the operation of a probe connected to said frontend subsystem by transmitting and receiving ultrasound wave into the target of interest, condition and digitize the received signal, and send the digital data into an imaging processor; and
a PC system configured to implement the image processor to generate at least one ultrasound image;
wherein the frontend subsystem is further configured to have a single or a plurality of frontend module(s) of the form of computer high-speed bus PCIe card(s) which is(are) plugged into a single or a plurality of high-speed PCIe expansion slot(s) of the PC system's PCIe expansion subsystem.
wherein said frontend module's PCIe form factor matches PCI slot of said PC system's PCIe expansion subsystem box and secures itself to said PC system's PCIe expansion subsystem box when plugged into the PCIe expansion slot of said PC system.

2. The ultrasound imaging apparatus of claim 1, wherein the frontend module is configured to has a connector sit on the PCIe bracket,
wherein said connector is used to connect to a probe outside of the PC case.

3. The ultrasound imaging apparatus of claim 1, wherein the frontend module is configured to have power input, clock and synchronization signal input or output.

4. The ultrasound imaging apparatus of claim 1, wherein the frontend module is configured to have a FPGA to control the transmit and receive of the ultrasound signals as well as sending the ADC data to the CPU or GPU through PCIe connector.

5. The ultrasound imaging apparatus of claim 4, wherein the frontend module is further configured to send per transmit/receive event ADC data to CPU or GPU memory.

6. The ultrasound imaging apparatus of claim 4, wherein the FPGA is a low end FPGA control unit with PCIe IP controller.

7. The ultrasound imaging apparatus of claim 4, wherein the FPGA is further configured to have a Soft CPU implemented on the FPGA to implement: a) transmit signal generation and excitation of a probe; b) receiving signal, conditioning, and ADC sampling; c) coordinating transmit and receive;
wherein the FPGA is further configured to have a soft CPU implemented on the FPGA to send ADC sampled data back to the imaging processor.

8. The ultrasound imaging apparatus of claim 7, wherein a dual port data buffer is used to work as a common buffer bridge between these two soft CPUs.

9. The ultrasound imaging apparatus of claim 1, wherein the frontend module is further configured to have an EM shielding structure to protect the sensitive circuit from EM interferences inside PC system's PCIe expansion subsystem box

10. The ultrasound imaging apparatus of claim 9, wherein the EM shielding structure and the PCI bracket are made out of one piece of metal.

11. The ultrasound imaging apparatus of claim 1, wherein the frontend module can be configured as either master or slave;
wherein the master frontend module is configured to output clock signal and synchronization signal;
wherein the slave frontend module can receive clock signal and synchronization signal.

12. The ultrasound imaging apparatus claim 1, further comprising: a probe adapter module with shielding box wherein the probe adapter connect to the frontend subsystem through the connector on the PCIe bracket of the frontend module;
wherein the probe adapter has a probe connector interface which is used to connect to a probe.

13. The ultrasound imaging apparatus claim 1, further comprising: a probe adapter subsystem which has multiple cables and connectors pairs where some cable/connector pair connects to the connector on the frontend module, some cable/connector pair connects to each individual probe, switches are used to selectively connect different probes to the frontend modules.

14. The ultrasound imaging apparatus of claim 1, wherein the image processor subsystem is further configured to have Pre-Allocate buffers in host memory for receiving data from frontend modules during the startup of the PC.

15. The ultrasound imaging apparatus of claim 1, wherein the PC system is configured to have a PSU module;
wherein the PSU can use medical grade PSU to meet medical regulations.

16. An ultrasound treatment apparatus comprising: a treatment frontend subsystem configured to control the operation of a probe connected to said frontend subsystem by transmitting ultrasound wave into the target of interest; and
a PC system configured to control the frontend subsystem;
wherein the frontend subsystem is further configured to have a single or a plurality of Frontend module(s) of the form of computer high-speed bus PCIe card(s) which is(are) plugged into a single or a plurality of high-speed PCIe expansion slot(s) of the PC system's PCIe expansion subsystem.
wherein said Frontend module's PCIe form factor matches PCI slot of said PC system's PCIe expansion subsystem box and secures itself to said PC system's PCIe expansion subsystem box when plugged into the PCIe expansion slot of said PC system.

17. An ultrasound treatment as well as imaging apparatus comprising: an imaging frontend subsystem configured to control the operation of a probe connected to said frontend subsystem by transmitting and receiving ultrasound wave into the target of interest, condition and digitize the received signal, and send the digital data into an imaging processor;
a treatment frontend subsystem configured to control the operation of a probe connected to said frontend subsystem by transmitting ultrasound wave into the target of interest; and
a PC system configured to implement the image processor to generate at least one ultrasound image;
wherein the frontend subsystem is further configured to have a single or a plurality of Frontend module(s) of the form of computer high-speed bus PCIe card(s) which is(are) plugged into a single or a plurality of high-speed PCIe expansion slot(s) of the PC system's PCIe expansion subsystem.
wherein said Frontend module's PCIe form factor matches PCI slot of said PC system's PCIe expansion subsystem box and secures itself to said PC system's PCIe expansion subsystem box when plugged into the PCIe expansion slot of said PC system.
